(19) United States
(12) Plant Patent Application Publication
(10) Pub. No.: US 2007/0107104 P1
(43) Pub. Date: May 10, 2007

(54) Hybrid tea rose plant named 'Meiwimova'

(76) Inventor: Alain A. Meilland, Antibes (FR)

Correspondence Address: BUCHANAN, INGERSOLL & ROONEY PC, POST OFFICE BOX 1404, ALEXANDRIA, VA, 22313-1404, US

(21) Appl. No.: 11/269,715

(22) Filed: Nov. 9, 2005

(51) Int. Cl. A01H 5/00

(52) U.S. Cl. PLT/133

(57) ABSTRACT

A new and distinct variety of Hybrid Tea rose plant is provided which abundantly forms on a substantially continuous basis attractive elegant long-lasting double white blossoms wherein the blossom petals of a mature blossom tend to enclose the central portion of the blossom. The new variety is a spontaneous mutation of unknown causation of the ‘Meivamo’ variety (U.S. Plant Pat. No. 8,619). The plant exhibits a vigorous and upright growth habit, and strong and vigorous dark green foliage with a glossy aspect. No particular susceptibility to crytogamic diseases has been observed. The attractive dark green foliage contrasts nicely with the pure white blossom coloration. The new variety is well suited for cut flower production under greenhouse growing conditions. Excellent forcing has been observed particularly during the winter.

### BOTANICAL/COMMERCIAL CLASSIFICATION

Rosa hybrida/Hybrid Tea Rose

### VARIETAL DENOMINATION

cv. Meiwimova

### SUMMARY OF THE INVENTION

The new variety of Rosa hybrida Hybrid Tea rose plant was discovered at LeLuc, France, while growing among plants of the ‘Meivamo’ variety (U.S. Plant Pat. No. 8,619, granted Mar. 1, 1994). The new variety is a spontaneous mutation of unknown causation of the ‘Meivamo’ variety. I was primarily attracted to the new variety because of its distinctive blossom coloration. Had the new variety not been discovered and preserved by me it would have been lost to mankind.

The ‘Meivamo’ variety is a spontaneous mutation of the ‘Keiromo’ variety (U.S. Plant Pat. No. 7,138).

As discussed in U.S. Plant Pat. No. 7,138, the parent ‘Keiromo’ variety was the product of a controlled breeding program. The female parent (i.e., the seed parent) of the ‘Keiromo’ variety was the ‘Jafitali’ variety (U.S. Plant Pat. No. 4,083) and the male parent (i.e., the pollen parent) was the ‘Happiness’ variety (U.S. Plant Pat. No. 911). The parentage of the parent ‘Keiromo’ variety can be summarized as follows:
‘Jofitali’×‘Happiness’.

It was found that the new variety of the present invention
(a) exhibits a vigorous and upright growth habit that is substantially identical to that of the ‘Meivamo’ variety (U.S. Plant Pat. No. 8,619),
(b) forms in abundance elegant long-lasting double white blossoms wherein the blossom petals of a mature blossom tend to enclose the central portion of the blossom unlike the ‘Meivamo’ variety,
(c) forms strong and vigorous dark green glossy vegetation that contrasts well with the white blossom coloration,
(d) is well adapted for greenhouse forcing to produce cut flowers, and
(e) is not particularly affected by crytogamic diseases.

The new variety well meets the needs of the horticultural industry and can be grown to advantage to produce cut flowers under greenhouse growing conditions. The blossoms are highly attractive and are formed in abundance on a substantially continuous basis. The ability of the plant to undergo forcing has been found to be excellent particularly during the winter.

The new variety can be readily distinguished from its ‘Meivamo’ parent. More specifically, the ‘Meivamo’ variety forms blossoms that are scarlet pink on the inside and light carmine rose on the outside. Also, the petals of the ‘Meivamo’ variety lack a propensity to enclose the central portion of the blossoms (i.e., the pistils and stamens) when the blossoms are mature.

Additionally, the new variety of the present invention can be readily distinguished from its ancestors that produced the parent ‘Meivamo’ variety. The ‘Jofitali’ variety forms rose-bengal colored blossoms with a cardinal red center, and the ‘Happiness’ variety forms blossoms that are medium red in coloration.

The new variety has been found to undergo asexual propagation in France and in the United States, by a number of routes, including budding, grafting, and cuttage. Asexual propagation by the above-mentioned techniques at LeLuc, France and Wasco, Calif., U.S.A. has shown that the characteristics of the new variety are stable and are strictly transmissible by such asexual propagation from one generation to another.

The new variety has been named ‘Meiwimova’.

### BRIEF DESCRIPTION OF THE PHOTOGRAPH

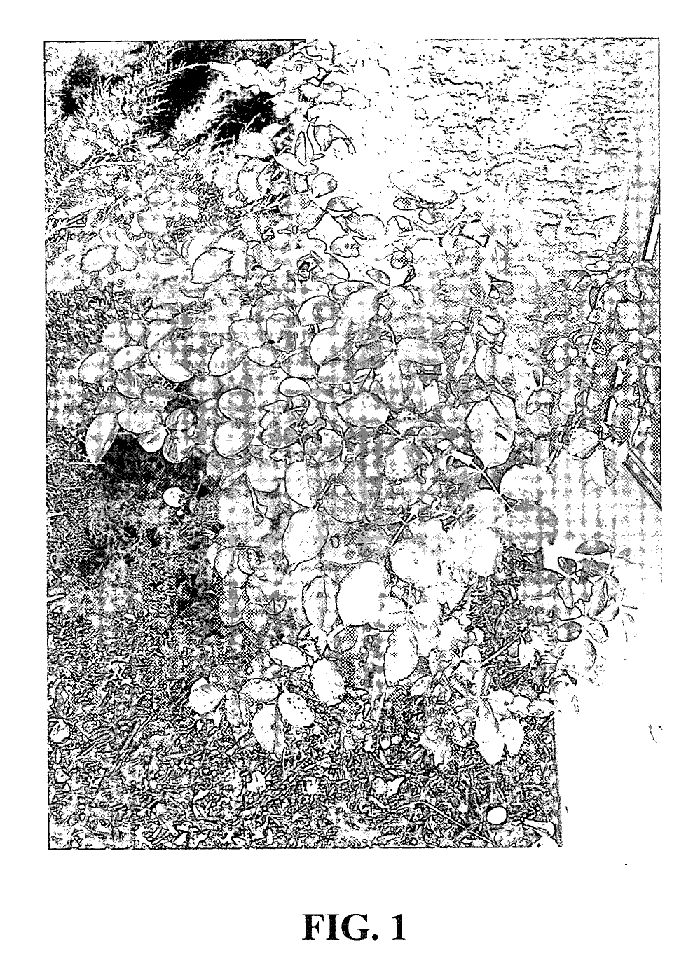

The accompanying photograph of FIG. 1 shows as nearly true as it is reasonably possible to make the same, in a color illustration of this character, a typical specimen of the new variety. The rose plant of the new variety was approximately three years of age and was photographed during early November while budded on Rosa indica understock and growing outdoors at West Grove, Pa., U.S.A. Budding took place in California and for the first two years the plant was grown outdoors in California prior to shipment to Pennsylvania.

### DETAILED DESCRIPTION

The chart used in the identification of the colors is that of The Royal Horticultural Society (R.H.S. Colour Chart). The description is based on the observation of two year-old plants during October while budded on Rosa indica understock and growing in greenhouses at West Grove, Pa., U.S.A.
Class: Hybrid Tea.
Plant:
   Height.—When pruned to a height of 85 cm, floral stems having a length of approximately 50 to 70 cm commonly are produced. When grown in the field at Wasco, Calif., U.S.A., a plant height of approximately 1.3 m commonly is observed at the end of the growing season.
   Habit.—upright.
Branches:
   Color.—young stems: light green, Green Group 143A and more or less shaded with some reddish coloration. — adult wood: medium green, Green Group 137B.
   Thorns.—size: average. — quantity: moderately numerous. — color: greenish on young stems and tan on mature wood.
Leaves:
   Stipules.—adnate, pectinate, fairly wide and linear.
   Petioles.—upper surface: striped reddish brown on young foliage and medium green with glandular edges on adult foliage. — under surface: light green and bear a few thorns.
   Leaflets.—number: 3, 5, and 7 (most often). — shape: generally oval. — serration: single and regular. — texture: firm. — general appearance: dense and glossy. — color (young foliage): upper surface: medium green, Green Group 137A. under surface: light green, Green Group 137C, and commonly widely tinted with reddish-brown coloration. — color (adult foliage): upper surface: dark green, Green Group 139A. under surface: light green, Green Group 137C.
Inflorescence:
   Number of flowers.—commonly one flower per stem.
   Peduncle.—rigid, relatively straight, light green in coloration, bears numerous light green aciculae, and the length commonly is approximately 11 to 12 cm on average.
   Sepals.—upper surface: tomentose and greenish in coloration. — under surface: light green in coloration and the edges of the outer sepals are glandular and slightly appendiculated.
   Buds.—shape: conical. — size: large. — length: approximately 4.5 cm on average. — color: upper surface: Red Group 43C. under surface: Red Group 43B.
   Flower.—shape: initially elongated, changing to cuplike with an apple-shaped center; and fully double. — diameter: approximately 12 to 13 cm on average. — color (when opening): upper surface: White Group 155C. under surface: White Group 155C. — color (when blooming): upper surface: White Group 155C. under surface: White Group 155C. — color (at end of blooming: upper surface: White Group 155C. under surface: White Group 155C. — fragrance: very slight. — lasting quality: long on the plant and when cut and placed in a vase. — petal number: approximately 32 to 35 on average under normal growing conditions. — petal arrangement: petals tend to enclose the central portion of the blossoms when mature unlike the parent ‘Meivamo’ variety. — petal drop: fairly good with the petals commonly detaching cleanly before drying. — stamen number: approximately 234 on average. — petal texture: consistent. — anthers: normal in appearance and yellowish in coloration. — pollen: present. — filaments: free-standing, bright yellow in coloration with fuschia tips, and commonly of irregular heights. — pistils: approximately 172 on average. — stigmas: normal in appearance, straw-colored, and commonly located below the stamens. — styles: free-standing, light fuschia in coloration, and commonly of irregular heights. — receptacle: light green in coloration, fairly smooth, and in longitudinal section in the shape of a pitcher.
Development:
   Vegetation.—strong and vigorous.
   Blooming.—abundant and substantially continuous when forced in a greenhouse for fresh-cut flower production.
   Resistance to diseases.—good with no particular susceptibility to crytogamic diseases having been observed.

1. A new and distinct Hybrid Tea rose plant characterized by the following combination of characteristics:
(a) exhibits a vigorous and upright growth habit that is substantially identical to that of the ‘Meivamo’ variety (U.S. Plant Pat. No. 8,619),
(b) forms in abundance elegant long-lasting double white blossoms wherein the blossom petals of a mature blossom tend to enclose the central portion of the blossom unlike the ‘Meivamo’ variety,
(c) forms strong and vigorous dark glossy green vegetation that contrasts well with the white blossom coloration, and
(d) is well adapted for greenhouse forcing to produce cut flowers, and
(e) is not particularly affected by crytogamic diseases;
substantially as herein shown and described.
